(19) United States
(12) Patent Application Publication
(10) Pub. No.: US 2014/0216449 A1
(43) Pub. Date: Aug. 7, 2014

(54) DEVICE FOR SURFACTANT ADMINISTRATION AND VENTILATION OF LOW BIRTH WEIGHT INFANTS

(71) Applicant: Los Angeles Biomedical Research Institute at Harbo-UCLA Medical Center, Torrance, CA (US)

(72) Inventor: Ruey-Kang Chang, Diamond Bar, CA (US)

(73) Assignee: Los Angeles Biomedical Research Institute at Harbor-UCLA Medical Center, Torrance, CA (US)

(21) Appl. No.: 14/135,331

(22) Filed: Dec. 19, 2013

Related U.S. Application Data
(60) Provisional application No. 61/847,232, filed on Jul. 17, 2013.
(60) Provisional application No. 61/739,637, filed on Dec. 19, 2012.

(51) Int. Cl. A61M 25/10; A61M 16/04

(52) U.S. Cl. CPC A61M 25/1011; A61M 16/0495; A61M 16/0434; A61M 2202/0488; A61M 2210/1039
USPC 128/202.16; 604/101.05; 128/207.15

(57) ABSTRACT

A fluid delivery and airway management device including a tubular member dimensioned for introducing a fluid into a trachea of a mammal, the tubular member having a proximal portion, a distal portion, and a middle portion between the proximal portion and the distal portion. The tubular member is dimensioned for positioning of the proximal portion in an oral cavity of a mammal, the middle portion in an oropharynx of the mammal and the distal portion in an esophagus of the mammal. An inflatable oral cavity balloon is positioned at the proximal portion and dimensioned to occlude the oral cavity. An inflatable esophageal balloon is positioned at the distal portion and dimensioned to occlude the esophagus. Apertures may be formed within the middle portion such that a fluid introduced into the tubular member is output through the apertures to a trachea.

### CROSS-REFERENCE TO RELATED APPLICATION

This application claims priority from U.S. Provisional Patent Application Ser. No. 61/847,232, filed on Jul. 17, 2013.

### FIELD

A device, kit and method for fluid delivery and/or airway management of a patient, more specifically, a device for surfactant delivery and ventilation of premature or otherwise very low birth weight infants. Other embodiments are also described herein.

### BACKGROUND

Many preterm infants suffer from respiratory distress syndrome (RDS) which can be caused by insufficient surfactant production and structural immaturity in the lungs. Such infants may therefore require surfactant replacement therapy. Surfactant replacement therapy refers to the administration of a surfactant to the infant's lungs and has been found to reduce mortality and morbidity rates in premature infants, reduce duration of ventilatory support, number of complications and medical costs. The surfactant is typically in liquid form and may be synthetic or animal derived.

The current standard practice of surfactant administration is to first intubate the premature infant with an endotracheal tube. The infant is then administered the surfactant in liquid form via the endotracheal tube. Next, the infant is extubated and subjected to nasal continuous positive air pressure (CPAP) to help drive the surfactant into the lungs. If the infant fails nasal CPAP, then he/she will be intubated again to start on mechanical ventilation via the endotracheal tube. Intubation of small, premature infants with an endotracheal tube, however, is a difficult procedure and therefore requires a clinician with a high degree of skill. In addition, endotracheal intubation can cause complications such as vocal cord injury, tracheal perforation and airway trauma.

Some new surfactant administration approaches in experimental stages include administering the liquid surfactant or an aerosolized surfactant nasally via CPAP. The effectiveness of nasal administration via CPAP, however, has not been demonstrated. In addition, since the pathway from the nose to the lungs is not sealed, some surfactant will enter into the mouth or esophagus, thus requiring higher surfactant doses (and increased cost). Moreover, although aerosolized administration may be promising, such approach is still experimental and therefore its efficacy is also in question.

### SUMMARY

The delivery method and device disclosed herein provides a secure, effective, and easily placed fluid (e.g. surfactant) administration and airway conduit for premature infants and other very low birth weight infants (VLBI) suffering from conditions such as respiratory distress syndrome (RDS). The device is designed to deliver a fluid such as a surfactant while the infant is receiving nasal CPAP support, and can also serve as a rescue airway when CPAP is not providing adequate ventilatory support. In this aspect, the airway device is configured to deliver surfactant, or air in cases where ventilator support is necessary, to the trachea without endotracheal intubation. Representatively, in one embodiment, the device includes a hollow tube dimensioned for insertion through the patient's mouth to the esophagus. An oral cavity balloon dimensioned to block the oral cavity is positioned at one end of the tube and an esophageal balloon dimensioned to block the esophagus is positioned at another, closed, end of the tube. Apertures are further provided in a side of the tube that is aligned with the oropharynx. In this aspect, when surfactant or air is delivered into the one end of the tube, it passes through the tube and out the apertures to the oropharynx. In the case of ventilatory support, a nose block may further be provided such that the only way for air pumped into the tube to go is out the apertures and to the trachea. In this aspect, the airway device allows for surfactant or air to be pumped directly into the trachea. Furthermore, the esophageal balloon prevents reflux of gastric content from causing aspiration. In addition, positioning of the oral cavity balloon in oral cavity, instead of the oropharynx, avoids compression of vital structures (nerve plexus, venous sinuses and carotid arteries).

### BRIEF DESCRIPTION OF THE DRAWINGS

The following illustration is by way of example and not by way of limitation in the figures of the accompanying drawings in which like references indicate like elements. It should be noted that references to “an” or “one” embodiment in this disclosure are not necessarily to the same embodiment, and such references mean at least one.

FIG. 1A illustrates a cross-sectional side view of one embodiment of a fluid delivery and airway management device.

FIG. 1B illustrates a cross-sectional side view of another embodiment of a fluid delivery and airway management device.

FIG. 2A illustrates a cross-sectional side view of one embodiment of the device of FIG. 1A or FIG. 1B.

FIG. 2B illustrates a cross-sectional side view of one embodiment of the device of FIG. 1A or FIG. 1B.

FIG. 3A illustrates a cross-sectional side view of one embodiment of the device of FIG. 1A or FIG. 1B.

FIG. 3B illustrates a cross-sectional side view of one embodiment of the device of FIG. 1A or FIG. 1B.

FIG. 4A illustrates a cross-sectional side view of another embodiment of a fluid delivery and airway management device.

FIG. 4B illustrates a cross-sectional side view of another embodiment of a fluid delivery and airway management device.

FIG. 5A illustrates a cross-sectional side view of one embodiment of the device of FIG. 4A or FIG. 4B.

FIG. 5B illustrates a cross-sectional side view of one embodiment of the device of FIG. 4A or FIG. 4B.

FIG. 6A illustrates a cross-sectional side view of one embodiment of the device of FIG. 4A or FIG. 4B.

FIG. 6B illustrates a cross-sectional side view of one embodiment of the device of FIG. 4A or FIG. 4B.

FIG. 7A illustrates a cross-sectional side view of one embodiment of the device of FIG. 4A or FIG. 4B.

FIG. 7B illustrates a cross-sectional side view of one embodiment of the device of FIG. 4A or FIG. 4B.

FIG. 8 is a block diagram illustrating one embodiment of a process for surfactant delivery.

### DETAILED DESCRIPTION

In this section we shall explain several preferred embodiments of this invention with reference to the appended drawings. Whenever the shapes, relative positions and other aspects of the parts described in the embodiments are not clearly defined, the scope of the invention is not limited only to the parts shown, which are meant merely for the purpose of illustration. Also, while numerous details are set forth, it is understood that some embodiments of the invention may be practiced without these details. In other instances, well-known structures and techniques have not been shown in detail so as not to obscure the understanding of this description.

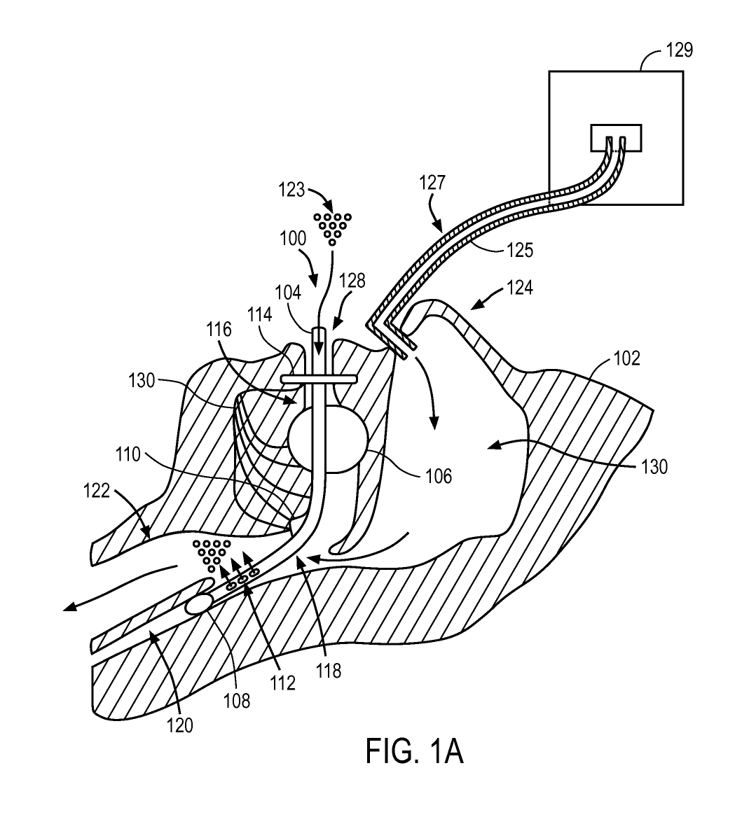

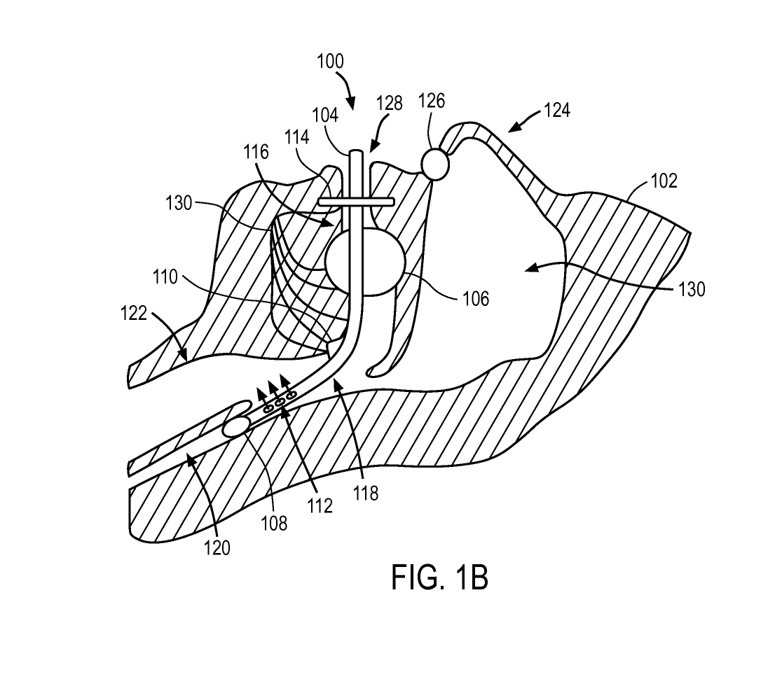

FIG. 1A illustrates a cross-sectional side view of one embodiment of a fluid delivery and airway management device positioned within an airway of a user. In one embodiment, device 100 may be positioned within an airway of a patient 102, which could be a mammal. Representatively, in one embodiment, device 100 is dimensioned for fluid delivery and/or management of an airway of a human patient. It is to be understood that the fluid suitable for delivery by device 100 may be any substance suitable for delivery within an air pathway of patient 102. The fluid may be any substance capable of flowing and changing shape in response to an applied pressure, for example, a substance in the form of a liquid, gas, aerosol or the like, that is suitable for delivery to an air pathway. For example, in the case of an infant suffering from RDS, the fluid may be a surfactant in a liquid or aerosol form. Alternatively, where device 100 is being used for airway management, the substance delivered may be air.

In one embodiment, device 100 may be dimensioned for use within a patient which may be a very low birth weight or premature infant, for example, weighing less than 1500 grams, more specifically from about 400 grams to about 1500 grams. In still further embodiments, device 100 is dimensioned for use within a newborn 30 days old or less. In other embodiments, device 100 may be dimensioned for use in an animal of any size and shape (e.g. a dog, a cat, a pig, a horse, a cow, etc.).

In some embodiments, device 100 may be several sizes depending upon the size of the patient. Representatively, in the case of a premature or very low birth weight infant, device 100 may have a first size for use in an infant less than about 700 grams, a second size for use in an infant from about 800 grams to about 1000 grams and a third size for use in an infant from about 1000 grams to about 1500 grams. In another embodiment, device 100 may have 2 sizes for premature infants-- a first size for use in an infant less than about 1000 grams, and a second size for use in an infant over 1000 grams. In the illustrated embodiment, patient 102 is a human.

As previously discussed, often times when the patient is a premature or an otherwise very low birth weight infant, their lungs are not fully developed and the infant is unable to produce a sufficient amount of surfactant necessary for proper lung function. Thus, it has been found that an air pathway to the lungs can be used to deliver additional surfactant to the infant's lungs. One representative air pathway is illustrated in FIG. 1A. Representatively, air passage to the lungs occurs when the individual breathes air in through nose 124 or mouth 128. In the case of the mouth, air passes from mouth 128, through oral cavity 116 and into the oropharynx 118, which is the oral part of the pharynx extending from the uvula to the hyoid bone. Air from nose 124 passes through nasal cavity 130 and also into oropharynx 118. From oropharynx 118, the pathway splits into the trachea 122, which extends to the lungs, and the esophagus 120, which extends to the stomach. Thus, in order to introduce a fluid (e.g. a surfactant or air) to the lungs, device 100 is dimensioned to create a substantially sealed air pathway from mouth 128 to trachea 122. Representatively, device 100 is dimensioned to deliver fluid 123 (e.g. a surfactant or air) to oropharynx 118 while blocking the esophagus 120 and fluid exits from nose 124 and mouth 128 such that the only way for the fluid 123 to go is from oropharynx 118 to the trachea 122 as indicated by the arrows.

To create such a sealed pathway, in one embodiment, device 100 includes tubular member 104, which is dimensioned to extend through mouth 128 to esophagus 108. In one embodiment, an end portion of tubular member 104 extending from mouth 128 includes one or more openings to allow for the introduction of fluid (e.g. a surfactant or air) and the other end is sealed to prevent air from exiting out the end and into esophagus 120. Apertures 120 are formed within a portion of tubular member 104 near the sealed end and within oropharynx 118 such that fluid introduced into the open end exits through apertures 120 toward trachea 122. Device 100 may further include an inflatable oral cavity balloon 106, which can be inflated within the oral cavity 116 to help position tubular member 104 within the air pathway of patient 102 and prevent fluid from exiting mouth 128 during a ventilation procedure. In addition, device 100 includes an inflatable esophageal balloon 108 positioned near the sealed end of tubular member 104, which can be inflated within or at an entrance to esophagus 120 to prevent the fluid from entering esophagus 120. In addition to preventing fluid entry, inflatable esophageal balloon 108 may be dimensioned to prevent reflux of gastric content from esophagus 120 without putting excessive pressure on the esophageal wall.

Device 100 may further include protrusion 110 which extends from a middle portion of tubular member 104 in a direction of tongue 130. Protrusion may be dimensioned to serve as a tongue holder which holds tongue 130 in place during inflation of oral cavity balloon 106 and prevents tongue 130 from posterior displacement thus blocking the air pathway to trachea 122. Air management device 100 may also include stabilizer 114. Stabilizer 114 may be positioned along a portion of tubular member 104 positioned to anchor the gum thus stabilizes the device 100 in the mouth.

In some embodiments, a nasal continuous positive air pressure (CPAP) device 127 may be used, which provides positive pressure to prevent fluid 123 escape from the nasal passage and drive fluid 123 into the lungs. Representatively, CPAP device 127 may include a nasal tube 125 positioned within nose 124 of patient 102. Nasal tube 125 may be connected to an air pressure machine 129 that outputs a positive air pressure through nasal tube 125. The air exits nasal tube 125 into the nasal passage 130 and travels through the previously discussed sealed air passage to the lungs as illustrated by the arrows. As the air travels through the air pathway toward the lungs, it intersects with any fluid 123 (e.g. a surfactant) within oropharynx 118 and drives fluid 123 into the lungs.

FIG. 1B illustrates a cross-sectional side view of another embodiment of a fluid delivery and airway management device positioned within an airway of a user. Device 100 is substantially similar to the device described in reference to FIG. 1A except in this embodiment, a nose block 126 may further be provided. Nose block 126 may be any type of nose blocking device such as a nose clip or other mechanism capable of sealing nose 124 and occluding the nostrils and preventing air exits through nose 124. It is further contemplated that in some embodiments, a pulse oximeter sensor or other similar sensing device may be integrated with, or placed near, the nose block 124 such that the oxygen saturation or other physiologic parameters of the patient can be monitored during a ventilation procedure. For example, nose block 126 could be used when a nasal CPAP device is not used, where the fluid administered through device 100 has already reached the lungs and CPAP ventilation is not required.

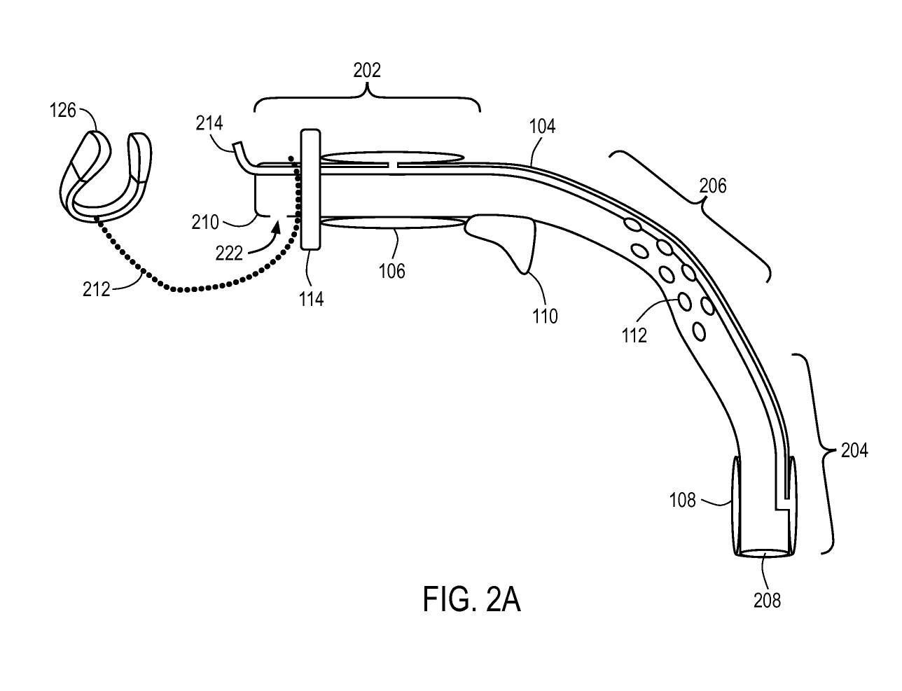

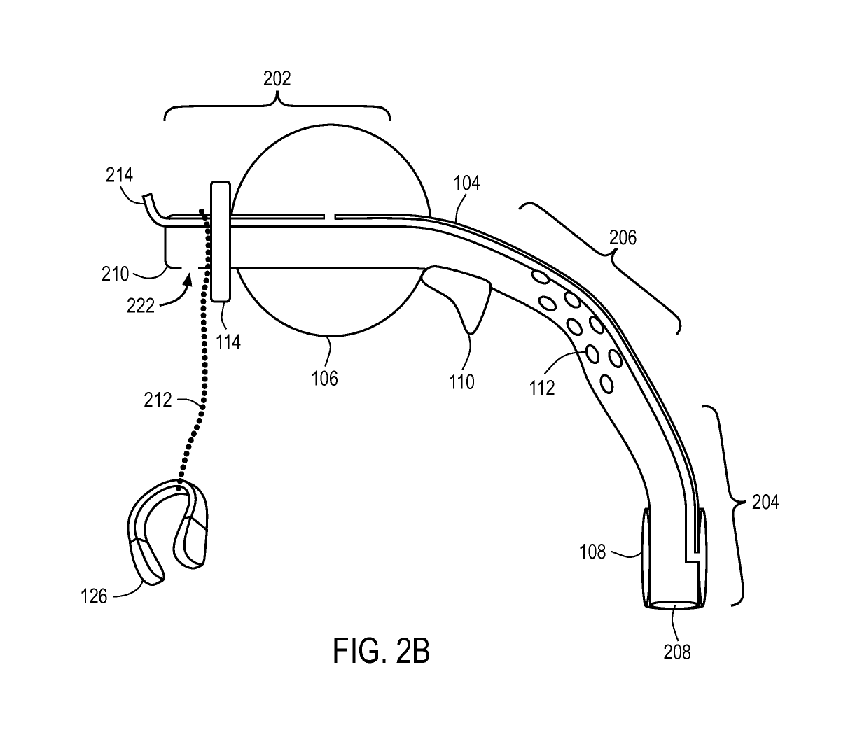

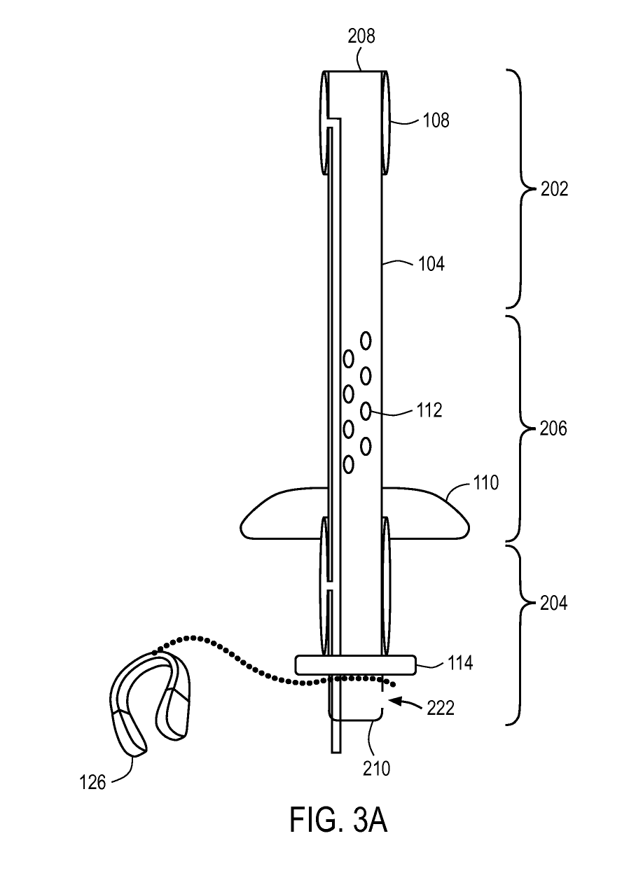

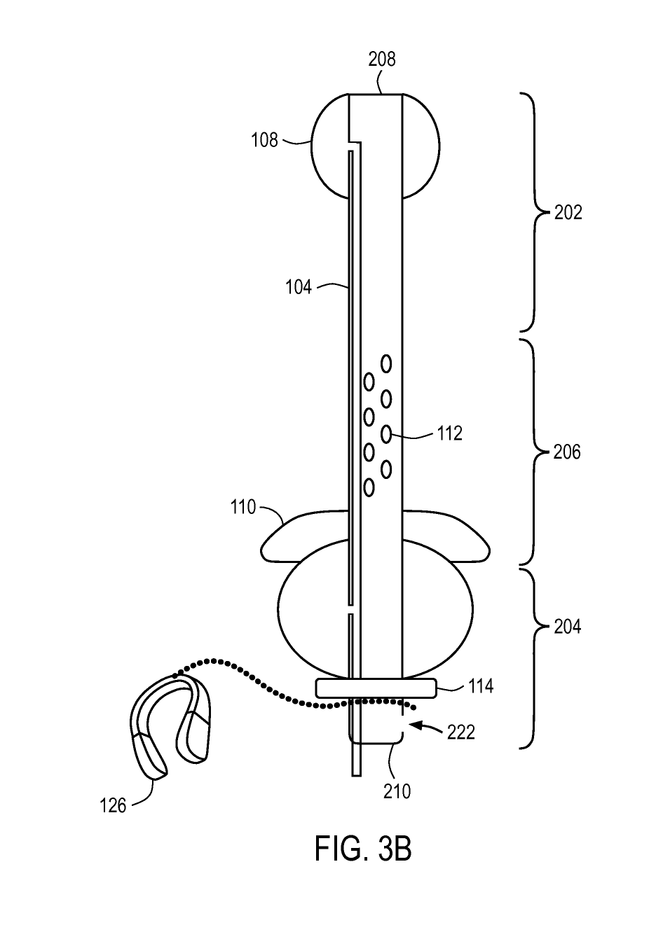

Each of the aspects of device 100 will now be described in further detail in reference to FIG. 2A, FIG. 2B, FIG. 3A and FIG. 3B. Referring to FIG. 2A and FIG. 2B, FIG. 2A illustrates a cross-sectional side view of device 100 in a deflated configuration and FIG. 2B illustrates a cross-sectional side view of device 100 in an inflated configuration. FIG. 3A illustrates a cross-sectional top view of device 100 in a deflated configuration and FIG. 3B illustrates a cross-sectional top view of device 100 in an inflated configuration.

Returning to FIG. 2A-2B, from this view it can be seen that tubular member 104 is a hollow tube having a proximal portion 202, a middle portion 206 and a distal portion 204. During use, proximal portion 202 is positioned within the oral cavity while distal portion 204 is positioned into the esophagus of the patient. Middle portion 206 of tubular member 104 may form a curve such that tubular member 104 substantially conforms to the structure of the air pathway of the patient and can be advanced through oral cavity 116 to esophagus 120. In some embodiments, open end 210 of tubular member 104 may have the dimensions of a universal connector used in endotracheal tubes for connection with a self-inflation bag device or ventilator.

In addition, proximal portion 202 may include a proximal port 222 through a side of tubular member 104. Port 222 may have any size and shape suitable for introducing a fluid into tubular member 104. Representatively, in one embodiment, port 222 may be sized such that a syringe containing a fluid such as a surfactant can be injected from the syringe, through port 222 and into tubular member 104. Once the surfactant is introduced into tubular member 104 through port 222, the self-inflation bag device or ventilator connected to open end 210 of tubular member 104 may be used to provide a positive air pressure sufficient to drive the surfactant down tubular member 104 and out apertures 112.

Port 222 may, however, be optional and, instead, the surfactant can be delivered into tubular member 104 through open end 210. Representatively, where port 222 is omitted, a surfactant or other fluid substance can be introduced into open end 210 of tubular member 104 using a syringe, or other similar delivery device. Once introduced into tubular member 104, the self-inflation bag device or ventilator can be connected to open end 210 to deliver a positive pressure into tubular member 104 and drive the surfactant through tubular member 104 and out apertures 112.

In some embodiments, tubular member 104 may be made of any semi-rigid material such as polyethylene or a clear polyvinyl chloride (PVC) suitable for insertion along an air passageway of a patient. In addition, in some embodiments, the diameter of tubular member 104 may taper toward sealed end 208 and the material used in the esophageal portion (i.e. distal portion 204) may be less rigid than other portions of tubular member 104 (e.g. middle portion 206 and/or proximal portion 202) to avoid esophageal injury.

Inflatable oral cavity balloon 106 may be mounted to proximal portion 202 of tubular member 104 so that when tubular member 104 is in place, oral cavity balloon 106 is positioned within oral cavity 116 as illustrated in FIG. 1A or FIG. 1B. In one embodiment, inflatable oral cavity balloon 106 may be positioned at a region of tubular member 104 and dimensioned such that it only occludes oral cavity 116 and does not occlude nasal cavity 130. In other words, oral cavity balloon 106 may be confined to the oral cavity 116 and does not extend to other regions such as the oropharynx 118, or other regions adjacent middle portion 206. Rather, oral cavity balloon 106 is positioned between stabilizer 114, and in some cases contacting stabilizer 114, and the bend portion of middle portion 206. Inflatable oral cavity balloon 106 may be substantially symmetric in the inflated configuration as shown. In other embodiments, oral cavity balloon 106 may be substantially asymmetric in the inflated configuration. Representatively, the distal end of oral cavity balloon 106 may have a larger diameter than the proximal end. This type of structure may help to compress and push the tongue forward such that oral cavity balloon 106 can also serve as a tongue holder. Alternatively, the distal end of oral cavity balloon 106 may have a smaller diameter than the proximal end to facilitate blocking of the oral cavity.

Oral cavity balloon 106 may be a substantially compliant balloon made of materials including, but not limited to, latex, polyurethane, nylon elastomers and other thermoplastic elastomers. In this aspect, oral cavity balloon 106 can be inflated until it fills the oral cavity and provides a seal in order to prevent fluid leak through the mouth. Oral cavity balloon 106 may be inflated and/or deflated by connecting a syringe (not shown) to inflation tube 214 which extends along tubular member 104 to oral cavity balloon 106. A connector at inflation tube 214 has a valve that opens when a syringe is connected, thus allows air to be injected to or withdrawn from the tube 214 and balloon 106. Injecting air via the syringe will in turn deliver air to oral cavity balloon 106 causing oral cavity balloon 106 to inflate. Oral cavity balloon 106 may be deflated by withdrawing air through inflation tube 214 using the syringe. In some embodiments, inflation tube 214 may extend through the lumen of tubular member 104 and through the wall to oral cavity balloon 106. Alternatively, inflation tube 214 may extend along the outside of tubular member 104.

In some embodiments, esophageal balloon 108 may also be connected to inflation tube 214. In this aspect, oral cavity balloon 104 and esophageal balloon 108 may be inflated or deflated at the same time or in sequence (by varying the resistance of balloons to allow esophageal balloon to fill up first then the oral cavity balloon). In other embodiments where independent inflation/deflation of esophageal balloon 108 is desired, a separate inflation tube may be connected to esophageal balloon 108. As previously discussed, esophageal balloon 108 is used to block the opening to esophagus 120 as illustrated in FIG. 1A and FIG. 1B. Esophageal balloon 108 may therefore be mounted to distal portion 204 of tubular member 204, near sealed end 208. Esophageal balloon 108 may be less compliant than oral cavity balloon 104 such that it can be inflated to a predetermined maximum size suitable for blocking an opening of the esophagus (e.g. to block acid reflux from the stomach) without putting excessive pressure on the esophageal wall. Representatively, in one embodiment, esophageal balloon 108 may be made of a polyethylene or other low-compliance polymer and have a maximum diameter which is substantially equal to that of the esophageal opening.

To facilitate positioning of oral cavity balloon 104 and esophageal balloon 108 at the desired region within the patient, tubular member 104 may have a length (and bend as previously discussed) such that when tubular member 104 is positioned within the patient, oral cavity balloon 104 is positioned within oral cavity 116 and esophageal balloon 108 is positioned within the superior portion of esophagus 120. Representatively, tubular member 104 may have any length and oral cavity balloon 104 and esophageal balloon 108 any dimension/shape suitable for positioning of device 100 within an airway path as described above for patients within any of the previously discussed age and size ranges. The dimensions and shape of tubular member 104, oral cavity balloon 104 and esophageal balloon 108 may also be suitable for use of the device 100 within a patient that is an animal (e.g. a horse, a cow, a pig, a dog, a cat, etc).

Protrusion 110 may extend from tubular member 104, near or within proximal portion 202 so that it is aligned with the tongue when air maintenance device 100 is positioned within the oral cavity. In some embodiments, protrusion 110 may have a substantially triangular profile with the distal portion being the base of the triangle and extending further from tubular member 104 farther than the proximal portion. In this aspect, the wider portion of protrusion 110 pushes the back portion of the tongue away from apertures 112 formed within proximal portion 206 so that it does not block apertures 112, or other air pathways.

Apertures 112 are formed within the middle portion 206 of tubular member 104 so that they are aligned within the oropharynx 118 (see FIG. 1A and FIG. 1B) of the patient when device 100 is in place. Although a plurality of apertures 112 are shown, it is contemplated that any number and diameter of apertures 112 suitable for outputting fluid to the trachea of the patient may be formed through tubular member 104. Representatively, in some embodiments, there may be only one of apertures 112 (e.g. one large aperture) while in another embodiment there is more than one of apertures 112 (e.g. a plurality of smaller apertures). In this aspect, when fluid (e.g. a surfactant or air) is pumped through tubular member 104, the fluid will flow through apertures 112 to the oropharynx. Since the exits to the mouth, nose and esophagus are sealed via oral cavity balloon 106, CPAP device 127 or nose block 126, and esophageal balloon 108, respectively, the pumped air will be forced by positive pressure to the trachea during inspiration. In addition, any expired air from the trachea can exit the trachea through tubular member 104 during expiration.

In some embodiments, nose block 126 may be attached to device 100 while in others nose block 126 may be separate from device 100 or omitted. Representatively, nose block 126 may be attached to device 100 by a chord 212 attached to the proximal portion 202 of tubular member 104 so that nose block 126 is near the patient's nose when device 100 is inserted within the patient's mouth. Once device 100 is in the desired position, nose block 126 can be positioned around the patient's nose to block air from exiting the nose. As previously discussed, nose block 126 may be any type of nose clip or other mechanism capable of restricting air passage through the patient's nose (e.g. a nose plug).

FIG. 3A and FIG. 3B illustrate top views of device 100 in the deflated and inflated configurations, respectively. From this view, it can be seen that protrusion 110 may have a width dimension greater than that of tubular member 104 such that it extends beyond the sides of tubular member 104. In some embodiments, protrusion 110 may have a width dimension substantially similar to that of the patient's tongue width such that it can hold a substantial portion of the tongue in the desired position without the sides of the tongue curling up. It can further be seen from this view that in some embodiments, apertures 112 can extend around a substantial portion of the circumference of tubular member 104. For example, apertures 112 may be formed within both the sides of tubular member 104 near or facing the trachea and the top of tubular member 104.

One representative way of using device 100 will now be described. For example, in one embodiment, device 100 having the appropriate dimensions for the patient is selected by the care provider. With both the oral cavity balloon 106 and esophageal balloon 108 deflated, tubular member 104 is placed within the patient's mouth and pointed posterior to prevent the tube from entering into the trachea. This part can be performed by properly placing the patient's head and opening the mouth manually without the use of a laryngoscope. Tubular member 104 is then advanced until protrusion 110 is aligned with the base of the tongue. A syringe (not shown) is connected to the inflation tube 214. Using the syringe, air is then pumped through inflation tube 214 and into oral cavity balloon 106 and esophageal balloon 108 until the oral cavity balloon 106 fills up and occludes the oral cavity so that air cannot exit. CPAP device 127 or nose block 126 may further be placed on the nose to block the nasal airway.

In embodiments where device 100 is used to deliver a fluid such as a surfactant to the lungs, the surfactant can be delivered into tubular member 104 through open end 210 or port 222, where provided, using a syringe or other similar delivery device.

Next, a self-inflation bag device or other device capable of providing positive pressure ventilation, is attached to the open end 210 universal connector of tubular member 104. The user then compresses the bag to pump air through tubular member 104 and drive the surfactant into the trachea via apertures 112. The steps of introducing the surfactant to tubular member 104 and introducing positive pressure may be repeated as necessary. For example, in some embodiments, it is desirable to deliver the surfactant to the lungs in separate doses. Thus, a first amount of the surfactant may be introduced into tubular member 104 and pumped into the lungs using a positive pressure. When open end 210 is connected to a self-inflation bag device and port 222 is connected to a syringe filled with surfactant fluid, the operator will inject the surfactant into port 222 first, followed immediately by pumping air through open end 210 by the bag device to optimize the delivery of surfactant to the lungs. Once the first amount reaches the lungs, a second amount of surfactant may be introduced into tubular member 104 and positive pressure applied again to drive the second amount of surfactant into the lungs.

In embodiments where device 100 is used primarily for ventilation, any one or more of the previously described steps can be followed with or without surfactant introduction. Successful placement of device 100 and adequate ventilation can be assessed by observing chest rise of the patient and auscultation of air movement using a stethoscope.

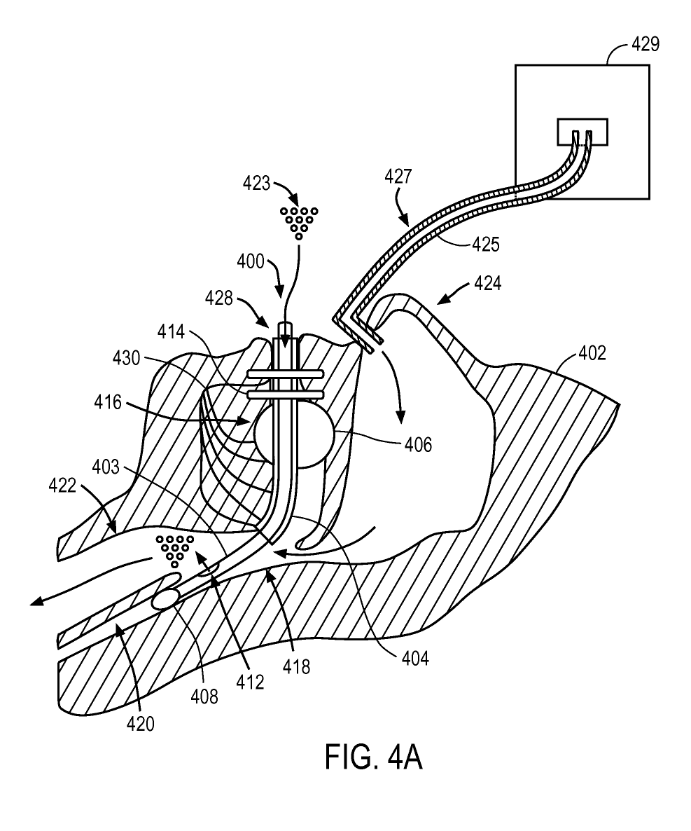

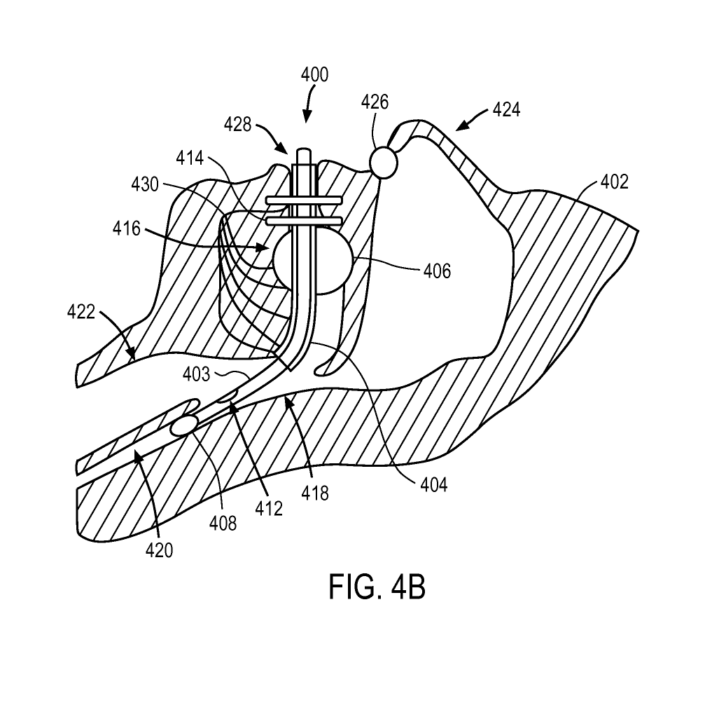

FIG. 4A illustrates a cross-sectional side view of another embodiment of a fluid delivery and airway management device positioned within an airway of a user. In one embodiment, device 400 may be positioned within an airway of a patient 402, which could be a mammal of any age and size as previously discussed in reference to FIG. 1A. Device 400 may be substantially similar to device 100 described in reference to FIG. 1A except that in this embodiment, device 400 includes an oral airway tube 404 and an esophageal tube 403 positioned concentrically inward of the oral airway tube 404. Oral airway tube 404 is dimensioned to pass from the mouth 428, through oral cavity 416 and to the base of the tongue 430. An inflatable oral cavity balloon 406 is attached to oral airway tube 404 so that in the inflated configuration, oral cavity balloon 406 can be used to block air exit from mouth 428. Esophageal tube 403 is dimensioned to extend through oral airway tube 404, from the mouth 428 to the esophagus 420. An inflatable esophageal balloon 408 is attached to the end of esophageal tube 403 near esophagus 420 and aperture 412 is formed within the portion of esophageal tube 403 positioned within the oropharynx 418. Similar to device 100, the patient's oral airway and nasal airway may be blocked using oral cavity balloon 416 and CPAP device 427, respectively, and the pathway to esophagus 420 blocked using esophageal tube 403 such that the only way for air pumped through esophageal tube 403 to go is out aperture 412 to trachea 422.

Device 400 may also include stabilizer 414. Stabilizer 414 may be positioned along a portion of oral airway tube 404 positioned near the gum so that if patient 402 bites down during the ventilation procedure, the force from the bite does not obstruct operation of device 400. Stabilizer 414 may further serve as a guide to help properly position device 400 within the patient 402.

In some embodiments, a nasal continuous positive air pressure (CPAP) device 427 may further be provided to seal the nasal passage and drive fluid 423 into the lungs. Representatively, CPAP device 427 may include a nasal tube 425 positioned within nose 124 of patient 102. Nasal tube 425 may be connected to an air pressure machine 429 that outputs a positive air pressure through nasal tube 425. The air exits nasal tube 425 into the nasal passage 130 and travels through the previously discussed sealed air passage to the lungs as illustrated by the arrows. As the air travels through the air pathway toward the lungs, it intersects with any fluid 423 (e.g. a surfactant) within oropharynx 118 and drives fluid 423 into the lungs.

In some embodiments, although not illustrated, an optional tongue holder may further be provided to hold tongue 430 in place during inflation of oral cavity balloon 406.

FIG. 4B illustrates a cross-sectional side view of another embodiment of a fluid delivery and airway management device positioned within an airway of a user. Device 100 is substantially similar to the device described in reference to FIG. 4A except in this embodiment, a nose block 426 may further be provided. Nose block 426 may be any type of nose blocking device such as a nose clip or other mechanism capable of sealing nose 424 and occluding the nostrils and preventing air exits through nose 424. It is further contemplated that in some embodiments, a pulse oximeter sensor or other similar sensing device may be integrated with, or placed near, the nose block 424 such that the oxygen saturation or other physiologic parameters of the patient can be monitored during a ventilation procedure. For example, nose block 426 could be used when a nasal CPAP device is not necessary, for example, where the fluid administered through device 400 has already reached the lungs and CPAP ventilation is not required.

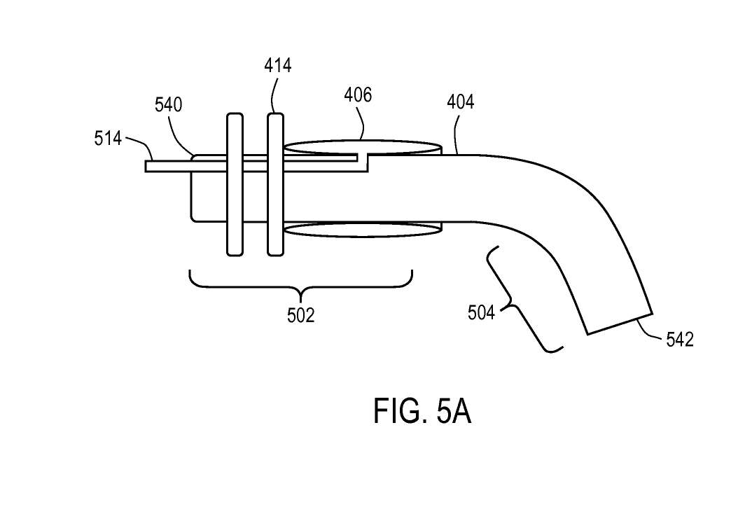

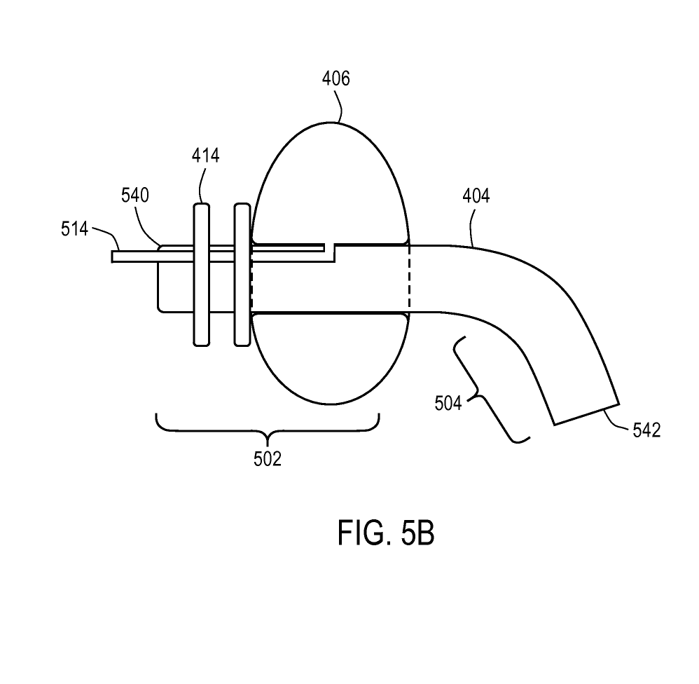

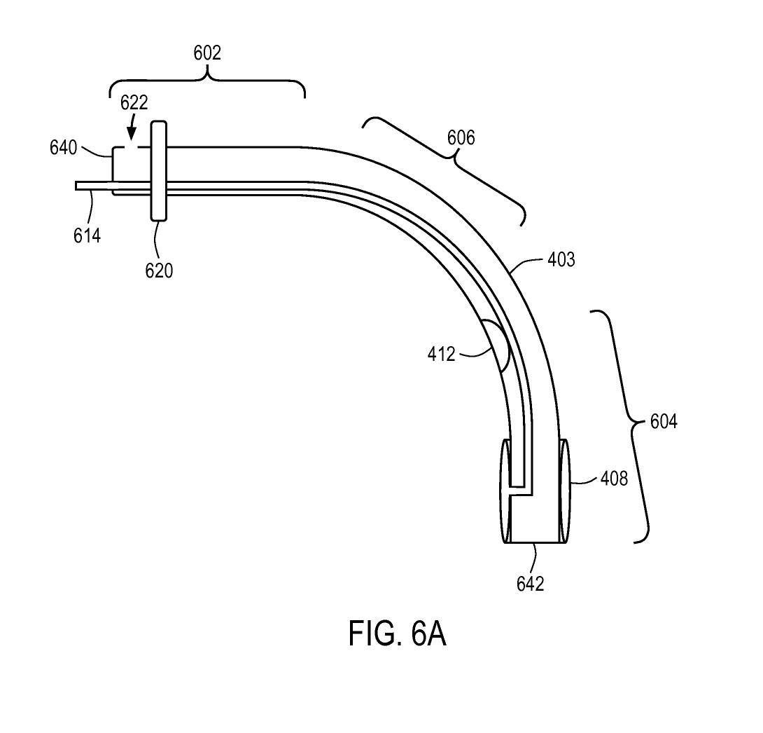

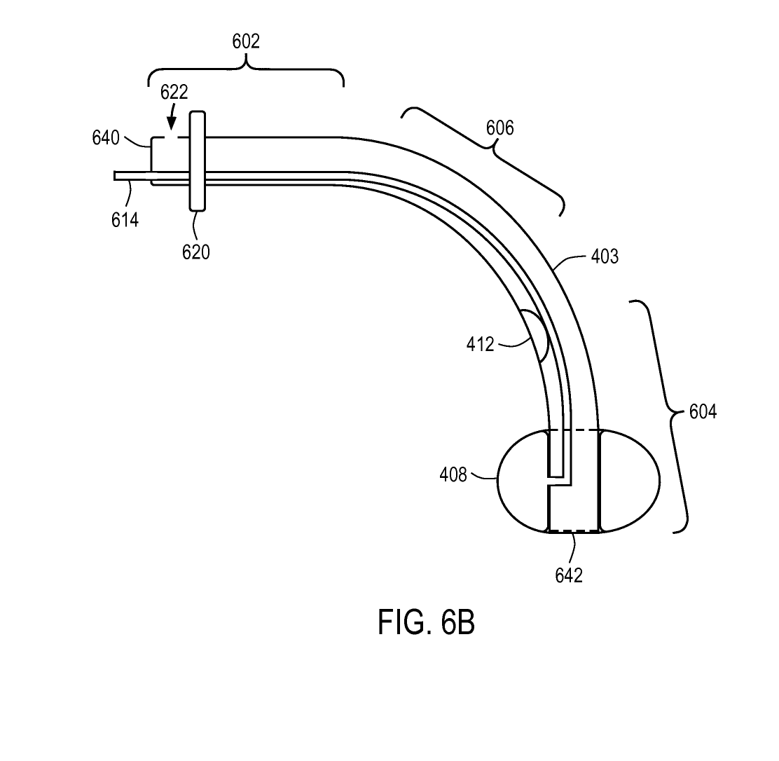

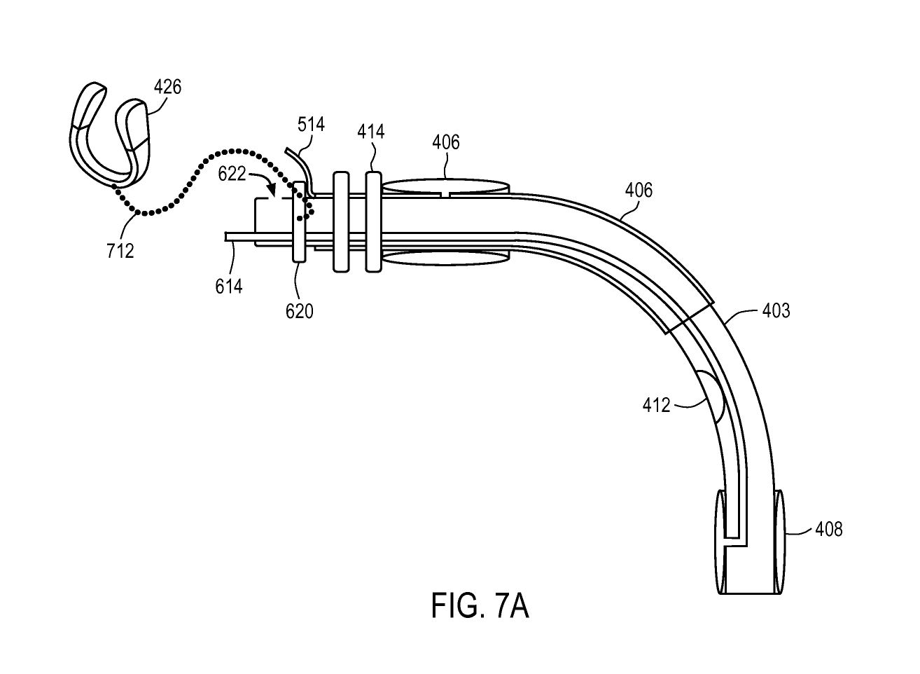

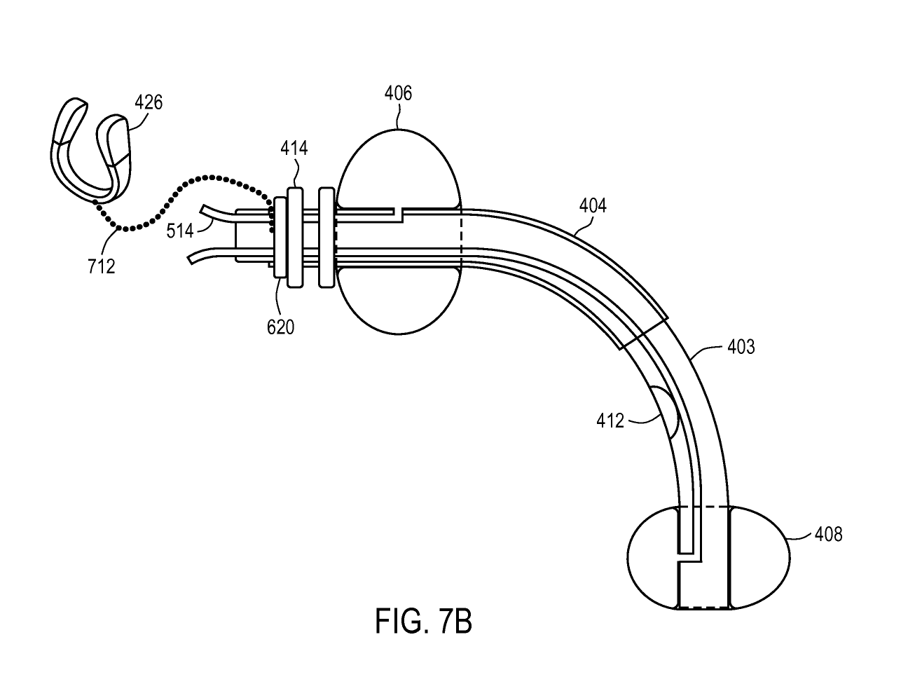

Each of the aspects of device 400 will now be described in further detail in reference to FIG. 5A, FIG. 5B, FIG. 6A, FIG. 6B, FIG. 7A and FIG. 7B. FIG. 5A and FIG. 5B illustrate cross-sectional side views of one embodiment of the oral airway tube of FIG. 4A and FIG. 4B in a deflated configuration and an inflated configuration, respectively. In one embodiment, oral airway tube 404 includes a proximal portion 502 terminating at a proximal end 540, and a distal portion 504 terminating at a distal end 542. When device 400 is positioned within the airway of the patient, proximal end 504 may be near mouth 428, and in some cases extend from mouth 428, while distal end 542 is positioned near the base of the tongue. Each of the proximal end 540 and the distal end 542 are open and oral airway tube 404 may have a lumen large enough to allow for insertion of esophageal tube 403 therethrough. Proximal end 540 can also be dimensioned to accommodate a universal adaptor that can be connected to a self-inflation bag device or other ventilating device. In some embodiments, oral airway tube 504 may be a semi-rigid tube made of, for example, polyethylene.

Oral cavity balloon 406 may be attached to the proximal portion 402 of oral airway tube 404 and positioned within the oral cavity of the patient during use. Oral cavity balloon 406 may be a substantially compliant inflatable/deflatable balloon having an outer diameter sufficient to fill the oral cavity and provide a substantially complete seal in order to prevent air leak via the mouth. In some embodiments, oral cavity balloon 406 may be an asymmetrical balloon such that when it is inflated, the proximal end diameter is greater than that of the distal end, or the distal end diameter is greater than that of the proximal end. Oral cavity balloon 406 may be made of any compliant material such as latex, polyurethane, nylon elastomers and other thermoplastic elastomers. Stabilizer 414 may be attached to the proximal portion 502 of oral airway tube 404 such that it is aligned with the gum of the patient when oral airway tube 404 is positioned within the patient's oral cavity.

Oral cavity balloon 406 may be inflated and/or deflated by connecting a syringe (not shown) to inflation tube 514 which extends along oral airway tube 404 to oral cavity balloon 406. Injecting air into the syringe will in turn deliver air to oral cavity balloon 406 causing oral cavity balloon 406 to inflate. Oral cavity balloon 406 may be deflated by withdrawing air through inflation tube 514 using the syringe. In some embodiments, inflation tube 514 may extend through the lumen of oral airway tube 404 and through the wall to oral cavity balloon 406. Alternatively, inflation tube 514 may extend along the outside of oral airway tube 404.

FIG. 6A and FIG. 6B illustrate cross-sectional side views of the esophageal tube of FIG. 4A and FIG. 4B in a deflated and an inflated configuration, respectively. Esophageal tube 403 includes a proximal portion 602 terminating at a proximal end 640, and a distal portion 604 terminating at a distal end 642. Esophageal tube 403 may further include a middle portion 606, between proximal portion 602 and distal portion 604, and having a bend so that esophageal tube 403 can conform to a shape of the air pathway of the patient. Esophageal tube 403 may have a length such that when device 400 is positioned within the airway of the patient, proximal end 604 may be near mouth 428, and in some cases extend from mouth 428, while distal end 642 is positioned near, or within, the esophagus 420. Proximal end 640 may be a substantially open end and the distal end 642 may be a sealed end such that air pumped into esophageal tube 604 can only exit through aperture 412. Proximal portion 602 may further include a proximal delivery port 622 through the side wall of tube 403 for introducing a fluid (e.g. a surfactant) into tube 403.

Esophageal tube 403 may have an outer diameter smaller than the inner diameter of the inner diameter of the oral airway tube 504 such that it can be inserted within and through oral airway tube 404. In some embodiments, when esophageal tube 403 is inserted through oral airway tube 504, proximal end 640 may be dimensioned to extend from the proximal end 540 of oral airway tube 504 and accommodate a universal adaptor that can be connected to a self-inflation bag device or other ventilating device. In some embodiments, esophageal tube 403 may be made of a clear PVC, or other similar material.

In some embodiments, esophageal balloon 408 is connected to the distal portion 604 of esophageal tube 403. An inflation tube 614, separate from inflation tube 514, may extend from the proximal end 602 to the distal end 604 and connect to esophageal balloon 408 to allow for inflation and deflation of esophageal balloon 408. Inflation tube 614 may run along the inner lumen of esophageal tube 403 or outside of esophageal tube 403. As previously discussed, esophageal balloon 408 is used to block the opening to esophagus 420 as illustrated in FIG. 4A. In some embodiments, esophageal balloon 408 may be less compliant than oral cavity balloon 404 such that it can be inflated to a predetermined maximum size suitable for blocking an opening of esophagus 420 (e.g. to block acid reflux from the stomach) without putting excessive pressure on the esophageal wall. Representatively, in one embodiment, esophageal balloon 408 may be made of a polyethylene or other low-compliance polymer and have a maximum diameter which is substantially equal to that of the esophageal opening.

Esophageal tube 403 may further include aperture 412 formed within distal portion 604. Aperture 412 may be a single opening or a plurality of openings formed through a portion of the wall of esophageal tube 403.

A stopper 620 may further be attached to the distal portion 602 of esophageal tube 403. Stopper 620 may be dimensioned to prevent proximal end 640 of esophageal tube 403 from being inserted through oral airway tube 404. In one embodiment, stopper 620 may be a ring shaped member which increases a diameter of oral airway tube 404. In this aspect, during an assembly operation, distal end 642 of esophageal tube 403 can be inserted through the proximal end 540 of oral airway tube 404 and pulled out the distal end 542 of oral airway tube 404 until stopper 620 reaches stabilizer 414 as illustrated in FIG. 7A and FIG. 7B.

FIG. 7A and FIG. 7B illustrate cross-sectional side views of the assembled device 400. From this view, it can be seen that when esophageal tube 403 is inserted through oral airway tube 404, oral airway tube 404 may overlap esophageal tube 403 along its proximal portion 602 and middle portion 606 such that the proximal end 640 and distal portion 604 of esophageal tube 403 are exposed. In this aspect, aperture 412 is positioned between the distal end 542 of airway tube 404 and the distal end 642 of esophageal tube 403, and exposed to the oropharynx (see FIG. 4). Since all the airway paths other than the trachea 422 are blocked by oral cavity balloon 404, esophageal balloon 408 and nose block 426, air exiting aperture 412 to the oropharynx 418 passes to trachea 422 and to the lungs. It is noted that in some embodiments, optional nose block 426 is attached to oral airway tube 404 or esophageal tube 403 via chord 712 as illustrated, while in other embodiments, nose block 426 is separated from device 400.

One representative way of using device 400 will now be described. For example, in one embodiment, the device 400 having the appropriate dimensions for the patient is selected by the care provider (e.g. EMT). Oral airway tube 404 and esophageal tube 403 may be inserted into the patients airway separated or as an assembled unit. For example, in one embodiment, oral airway tube 404 is first inserted into the patient's oral cavity followed by insertion of esophageal tube 403 through oral airway tube 404. Alternatively, esophageal tube 403 is inserted through oral airway tube 404 prior to positioning within the patient, and then the two together are inserted within the patient's mouth as a preassembled unit. In either case, both the oral cavity balloon 406 and esophageal balloon 408 are deflated prior to insertion of the tubing and then inflated once oral cavity balloon 406 is within the oral cavity and esophageal balloon 408 is within, or near the esophagus. Nose block 426 may then be placed on the nose to block the nasal airway.

A syringe (not shown) is connected to the inflation tubes 514 and 614. Using the syringe, air is then pumped through inflation tubes 514 and 614 and into oral cavity balloon 406 and esophageal balloon 408, respectively, until the oral cavity balloon 406 completely occludes the oral cavity so that air cannot exit. Connectors at inflation tubes 514 and 614 have a valve that opens when a syringe is connected, thus allows air to be injected to or withdrawn from the tubes 514 and 614 and balloons 406 and 408.

In embodiments where device 400 is used to deliver a fluid such as a surfactant to the lungs, the surfactant is introduced into tube 403 through port 622. A self-inflation bag device, or other device capable of providing positive pressure ventilation, is attached to the proximal end 640 of esophageal tube 403. The care provider then introduces a positive air pressure into tube 403 to drive the fluid through tube 403 and/or ventilate the patient by compressing the bag.

It is to be understood that any of the above described devices can be packaged as a kit with each of the parts pre-assembled or unassembled and the balloons deflated. The kit may come in a variety of different sizes to accommodate a variety of different patients. For example, in one embodiment, the device may be manufactured in three different sizes to accommodate a premature or otherwise very low birth weight infant within the weight ranges of (1) up to 700 grams, (2) about 700 g to about 1000 grams and (3) about 1000 grams to about 1500 grams. In still further embodiments, device 100 may have 2 sizes for premature infants-- a first size for use in an infant less than about 1000 grams, and a second size for use in an infant over 1000 grams.

It is further to be understood that any of the above described devices can be used to deliver a sufficient amount of surfactant continuously or serially in the absence of endotracheal intubation. Thus, the devices disclosed herein provide an effective and safe surfactant delivery system which requires much lower skills of the operator and avoids many complications associated with endotracheal intubation. Representatively, in one embodiment where the device is used for surfactant delivery, the care provider performs the following steps:

First, the appropriate sized device is selected based upon the size of the infant. Next, the infant is positioned supine with mouth open, the oropharynx is cleared, and nasal CPAP device is placed on the infant as needed. The device is then gently inserted into the esophagus. A syringe for inflating the balloons is connected to the inflation port followed by inflation of the esophageal cuff and oral cavity balloon until visually the balloon fills up the oral cavity with a seal around the cheek. The surfactant is then delivered into the tube (e.g. using a syringe). A self-inflation bag device is then connected to the tube and compressed to deliver a flow of air into the tube and drive the surfactant out the apertures toward the lungs. The device may be safely left in place as the infant continues on nasal CPAP. Thus, if the infant's respiratory status worsens despite the use of nasal CPAP, the care provider can use the device to connect to the bag-valve device or ventilator to deliver positive pressure ventilation.

In some embodiments, the surfactant is delivered in multiple doses or repeat doses and at a frequency dependent upon the clinical status of the patient. For example, in some embodiments, the surfactant is delivered in 6 to 24 hour intervals. It is noted that since the devices disclosed herein provide a substantially sealed delivery pathway to the lungs, as opposed to other methodologies such as nasal administration, the number of doses, frequency, and in some cases, dosage amount, may be reduced below that typically administered because substantially all of the surfactant reaches the lungs.

The surfactant may be any approved surfactant which mimics pulmonary surfactant. Representatively, the surfactant may be a natural exogenous surfactant or a synthetically manufactured surfactant. Representatively, the surfactant may be in fluid or in aerosol forms. Representative surfactants may include, but are not limited to, poractant alfa, calfactant, beractant and lucinactant. Representative doses may include, but are not limited to, from about 100-200 mg/kg/dose (1.25-2.5 mL/kg), 105 mg/kg/dose (3 mL/kg), 100 mg/kg/dose (4 mL/kg) and 5.8 mL/kg.

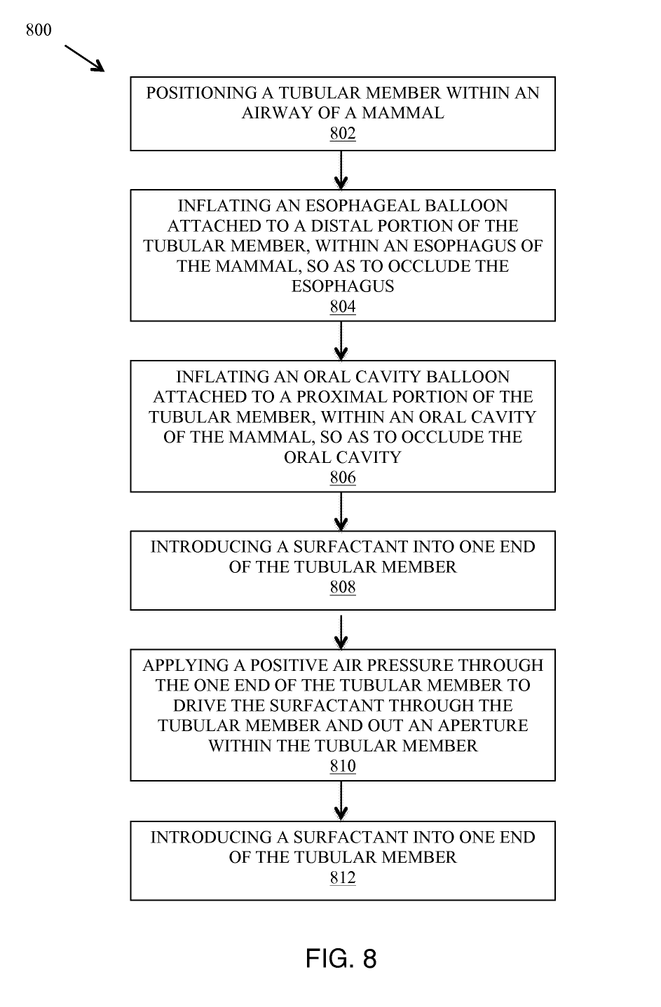

FIG. 8 is a block diagram illustrating one embodiment of a process for surfactant delivery. In one embodiment, process 800 may include positioning a tubular member within an airway of a mammal (block 802). The tubular member may be, for example, any of the previously discussed tubular members such as tubular member 104 discussed in reference to device 100. Process 100 may further include inflating an esophageal balloon attached to a distal portion of the tubular member, within an esophagus of the mammal, so as to occlude the esophagus (block 804). The esophageal balloon may be, for example, esophageal balloon 108 described in reference to device 100. In addition, an oral cavity balloon attached to a proximal portion of the tubular member, within an oral cavity of the mammal, may be inflated so as to occlude the oral cavity (block 806). The oral cavity balloon may be, for example, oral cavity balloon 106 described in reference to device 100. A surfactant may then be introduced into one end of the tubular member (block 808). Next, a positive air pressure may be applied through the one end of the tubular member to drive the surfactant through the tubular member and out an aperture within the tubular member (block 810). In addition, an air flow may be delivered into a trachea of the mammal to drive the surfactant toward a lung (block 812).

It is to be understood that in the case of fluid delivery, specifically surfactant delivery, the devices disclosed herein provide several advantages including: 1) more secure pathway for surfactant delivery; 2) a temporary rescue airway for premature and very low birth weight infants; 3) fewer injuries as compared to endotracheal intubation techniques which can cause vocal cord injury, tracheal perforation and airway trauma; 4) faster surfactant delivery; 5) more efficient surfactant delivery (e.g. a lower dosage can be used since the delivery pathway is directly to the lungs); and 6) lower skill than endotracheal intubation.

In the preceding detailed description, specific embodiments are described. It will, however, be evident that various modifications and changes may be made thereto without departing from the broader spirit and scope of the claims. The specification and drawings are, accordingly, to be regarded in an illustrative rather than restrictive sense.

What is claimed is:

1. A fluid delivery apparatus comprising:
a tubular member dimensioned for introducing fluid into a trachea of a mammal, the tubular member having a proximal portion, a distal portion, and a middle portion between the proximal portion and the distal portion, wherein the tubular member is dimensioned for positioning of the proximal portion in an oral cavity of a mammal, the middle portion in an oropharynx of the mammal and the distal portion in an esophagus of the mammal;
an inflatable oral cavity balloon positioned at the proximal portion and dimensioned to occlude the oral cavity;
an inflatable esophageal balloon positioned at the distal portion and dimensioned to occlude the esophagus; and
apertures formed within the middle portion such that fluid introduced into the tubular member is output through the apertures to a trachea.

2. The apparatus of claim 1 wherein the fluid is a surfactant.

3. The apparatus of claim 1 further comprising a fluid inlet port formed through a side of the proximal portion.

4. The apparatus of claim 1 further comprising:
a protrusion extending from the middle portion and dimensioned to hold a tongue at a desired position.

5. The apparatus of claim 1 further comprising:
a nose block device.

6. The apparatus of claim 1 further comprising:
an inflation tube in communication with the inflatable oral cavity balloon and the inflatable esophageal balloon so as to allow for inflation of the inflatable oral cavity balloon and the inflatable esophageal balloon.

7. The apparatus of claim 1 wherein the inflatable oral cavity balloon is asymmetric and dimensioned to both occlude the oral cavity and hold a tongue at a desired position.

8. The apparatus of claim 1 wherein the inflatable esophageal balloon is dimensioned to occlude an entire lumen of the esophagus and prevent reflux of gastric content out of the lumen.

9. A fluid delivery apparatus comprising:
an oral airway tube having a proximal end and a distal end, the oral airway tube having an inflatable oral cavity balloon positioned near the proximal end; and
an esophageal tube positioned concentrically inward of the oral airway tube, the esophageal tube having a proximal end extending from the proximal end of the oral airway tube and a distal end extending from the distal end of the oral airway tube, and wherein an inflatable esophageal balloon is positioned near the distal end, an opening is formed through a portion of the esophageal tube proximal to the inflatable esophageal balloon and a fluid inlet port is formed through a side of the esophageal tube, near the proximal end.

10. The apparatus of claim 9 wherein the oral airway tube and the esophageal tube are movable with respect to one another.

11. The apparatus of claim 9 further comprising:
a protrusion extending from the oral airway tube and dimensioned to hold a tongue at a desired position.

12. The apparatus of claim 9 further comprising:
a nose clip.

13. The apparatus of claim 9 further comprising:
an inflation tube in communication with the inflatable oral cavity balloon and the inflatable esophageal balloon so as to allow for inflation of the inflatable oral cavity balloon and the inflatable esophageal balloon.

14. The apparatus of claim 9 wherein the inflatable oral cavity balloon is asymmetric and dimensioned to both occlude the oral cavity and hold a tongue at a desired position.

15. The apparatus of claim 9 wherein the inflatable esophageal balloon is dimensioned to occlude an entire lumen of the esophagus and prevent reflux of gastric content out of the lumen.

16. A kit comprising:
a fluid delivery device dimensioned for introducing fluid into a trachea of a mammal, the fluid delivery device having a tubular member, an inflatable oral cavity balloon, an inflatable esophageal balloon and fluid delivery apertures formed within the tubular member for delivery of a fluid to a trachea; and
a continuous positive air pressure mechanism configured to deliver a positive air pressure to the trachea and drive the fluid from the trachea to the lungs.

17. A method for surfactant delivery comprising:
positioning a tubular member within an airway of a mammal;
inflating an esophageal balloon attached to a distal portion of the tubular member, within an esophagus of the mammal, so as to occlude the esophagus;
inflating an oral cavity balloon attached to a proximal portion of the tubular member, within an oral cavity of the mammal, so as to occlude the oral cavity;
introducing a surfactant into one end of the tubular member;
applying a positive air pressure through the one end of the tubular member to drive the surfactant through the tubular member and out an aperture within the tubular member; and
delivering an air flow into a trachea of the mammal to drive the surfactant toward a lung.

18. The method of claim 17 wherein the tubular member comprises an inner tubular member and an outer tubular member, and wherein positioning the tubular member comprises:
positioning the outer tubular within the oral cavity of the mammal; and
positioning the inner tubular member within the outer tubular member.

19. The method of claim 17 wherein the surfactant is a first amount of surfactant, the method further comprising:
after delivering an air flow, introducing a second amount of surfactant into the one end of the tubular member;
applying the positive pressure to drive the second amount of surfactant out the aperture; and
delivering the air flow to drive the second amount of surfactant toward the lung.
